(19) United States
(12) Patent Application Publication
(10) Pub. No.: US 2006/0175427 A1
(43) Pub. Date: Aug. 10, 2006

(54) Injection apparatus comprising a position sensor

(76) Inventors: Berthold Jonientz, Rueegsauschachen (CH); Philippe Kohlbrenner, Kaltacker (CH)

Correspondence Address: DORSEY & WHITNEY LLP;INTELLECTUAL PROPERTY DEPARTMENT, 50 SOUTH SIXTH STREET, MINNEAPOLIS, MN, 55402-1498, US

(21) Appl. No.: 11/327,282

(22) Filed: Jan. 6, 2006

Related U.S. Application Data
(63) Continuation of application No. PCT/CH04/00404, filed on Jun. 28, 2004.

(30) Foreign Application Priority Data
Jul. 9, 2003 (DE) 103 30 984.5

(51) Int. Cl. A01G 27/00; B67D 5/08; B05B 1/30; B05B 1/34

(52) U.S. Cl. 239/69; 239/533.1; 239/67; 239/71

(57) ABSTRACT

A delivery device, such as an injection apparatus, for delivering, administering or dispensing a substance, the delivery device comprising an operational mechanism and at least one passive, non-contact sensor for sensing a position associated with the operational mechanism.

### CROSS-REFERENCE TO RELATED APPLICATION(S)

This application is a continuation of International Patent Application No. PCT/CH2004/000404, filed on Jun. 28, 2004, which claims priority to German Application No. DE 103 30 984.5, filed on Jul. 9, 2003, and the entire content of both applications is incorporated herein by reference.

### BACKGROUND

The present invention relates to devices for delivering, administering or dispensing substances, and to methods of making and using them. More particularly, the present invention relates to medical devices, such as injection devices and infusion apparatus, having a sensor for identifying or detecting the position of an element of the device or apparatus, such as the position of a setting element for setting a dosage or amount of a substance to be administered.

Injection apparatus, including so-called injection pens, are used in many areas of medicine for administering a medical or pharmaceutical product such as insulin, hormone preparations, and other substances and/or medicants capable of injection. Infusion apparatus, such as an insulin pump, enable a substance to be repeatedly dispensed in a monitored way. Such delivery devices, whether in the form of an injection pen, syringe, infusion pump, etc, include various mechanical means, e.g., administering or dosing means, to set or select a particular product dosage and dispense it as exactly as possible from the device. In order to be able to monitor the administering process and its accuracy, sensors or probes are arranged within the apparatus which detect the movement of various elements of the mechanical means. From this, the setting of the mechanical means is ascertained, for example by means of a microprocessor, ASIC, chip or a suitable circuit, and may be indicated on the injection or infusion apparatus by a mechanical or electronic display.

Mechanical scanning is susceptible to contamination, moisture and wear and exhibits large tolerances between the individual elements. This restricts the accuracy in measuring the setting of an injection apparatus. Therefore, non-contact methods for determining the setting of such an apparatus have been developed. In one example, a number of sensors or measuring devices are arranged at various points on the apparatus, which are suitable for measuring the setting without the elements coming into contact with the measuring devices or sensors.

EP 1 095 668 A1 discloses an electronic administering pen for medical purposes wherein, to measure the setting of an administering means of the pen, the linear position of a helical rod of the administering mechanism or the rotational position of a setting button of a dosing means is measured. According to this example of an injection device, an optical code converter including a code disc coupled to the rotational movement of the setting button may be used. The rotational movement of the code disc is measured by an optical receiver. A microprocessor converts the number of rotations by the code disc into a dosage amount corresponding to the setting. Another sensor may be provided between the windings of the helical rod of the administering means and registers the movement in the longitudinal direction along the longitudinal axis of the pen. The administered amount of a product is determined from the shift of the helical rod. The two sensors operate independently of each other and each determines only one movement direction of a mechanical means of the pen.

While such measuring means may increase the accuracy in measuring a setting as compared to mechanical scanning, the arrangement of the individual parts of such measuring means within the apparatus is complex, such that manufacturing the apparatus is complicated and expensive. In addition, the circuitry and measuring methods of these measuring means are susceptible to moisture and vibrations. Accommodating the individual parts of the measuring means, such as the sensors and the counter pieces for the sensors, often requires structural changes in the injection or infusion apparatus, making it unnecessarily large or even restricting the other mechanisms of the apparatus.

WO 02/064196 A1 discloses an injection apparatus controlled by a closed switch unit comprising integrated sensors which monitor selected parameters of the apparatus. The closed switch unit is fixed within the injection apparatus. At least two pairs of integrated Hall elements are used as the sensors. The Hall elements co-operate with a magnetized ring which alternately exhibits north and south poles. The ring is arranged within a dosing means and is moved around the longitudinal axis of the injection apparatus in accordance with a rotational movement for setting a product dosage. In order to measure the volume of a dosage setting, it is necessary to determine the rotational movement of the magnetic ring relative to the closed switch unit. For this example of the injection apparatus, the Hall elements are arranged on a circular arc opposing the magnetic ring, in a defined arrangement with respect to each other and the magnetic ring. When movement is started, a start angle is defined and, on the basis of measuring the magnetic field during the movement of the magnetic ring relative to the Hall elements, an end angle is determined once the movement is terminated. The start and end angles and the measured magnetic field are compared with a stored table and a product dosage set is determined from the comparison.

Using optical receivers or Hall sensors, however, requires an energy source in order to establish whether the dosage setting has been altered, i.e. a Hall sensor has to be activated and a signal processed, transmitted, received and evaluated using stored energy. The typical injection apparatus includes a limited energy source, such as a battery. As a result, when a Hall sensor uses energy from the limited energy source, the service life of such an injection apparatus is shortened.

### SUMMARY

It is an object of the present invention to provide a delivery device, such as an injection device or infusion pump, wherein the position of a mechanism associated with the device can be easily and cost-effectively sensed, measured and/or determined.

In one embodiment, the present invention comprises a delivery device for delivering, administering or dispensing a substance, the delivery device comprising an operational mechanism and at least one passive, non-contact sensor for sensing a position associated with the operational mechanism. In some embodiments, the at least one sensor comprises at least two passive, non-contact magnetic sensors. In some embodiments, the delivery device further comprises at least a first magnetic body coupled to the operational mechanism, wherein the magnetization of the first magnetic body changes at least once in a selected direction relative to the magnetic body and, in some embodiments, the delivery device further comprises one of a second magnetic body or an FE punched bent part coupled to the delivery device.

In some embodiments, the present invention involves administering, delivering or dispensing devices such as injection devices. Typically, injection devices comprise various operational mechanisms and/or mechanical means such as an administering or dosing means constructed from a number of elements or components, at least some of which are moved relative to each other when the device is operated. For example, to administer a product from an injection apparatus, a sliding element such as a toothed rod is moved along the longitudinal axis of the apparatus relative to a product container, an apparatus casing or other guiding elements. A dosing means for setting a dosage volume to be administered may include a rotational element which is rotated relative to the casing or a threaded rod. In accordance with the present invention, in addition to these and/or other operational or functional structures or mechanisms, the injection apparatus comprises a measuring means which measures the setting of a mechanism of the apparatus and, therefore, of the setting or state of the injection apparatus, by sensing, assessing or determining the movement of elements relative to each other.

In one embodiment, the present invention comprises an injection device for administering a substance, the injection device comprising an operational mechanism and at least one passive, non-contact sensor for sensing a position associated with the operational mechanism.

One complexity addressed by certain embodiments of the present invention is conservation of energy in injection and infusion devices when setting and measuring delivery doses. According to one embodiment, an injection apparatus includes at least one passive, non-contact sensor which can generate and output signals for detecting the position of a setting element. The detected position may be a rotational position of a rotational setting element in an injection apparatus, for example.

According to some embodiments of the present invention, a passive component such as a magnetic switch or Reed contact may be used as the sensor, as opposed to using active components, such as optical recorders or Hall sensors. According to some embodiments of the present invention, no power flows when the passive sensor is in its resting state due to the circuit being interrupted by the magnetic switch or Reed contact. The one or more passive, non-contact sensors used in accordance with one embodiment of the invention may be installed in an injection apparatus so that in a resting state, a circuit may be interrupted by the sensor and/or magnetic switch or Reed contact, and accordingly no energy or only a little energy is consumed. According to this embodiment, the interrupted circuit is only closed by being activated, such as by changing the magnetic field acting on the sensor. Accordingly, the passive, non-contact sensor may generate digital signals, i.e. ON and OFF, which switch on or activate a measuring circuit and switch it off again, in order to detect the position of a setting element by counting the switching-on and switching-off processes. Using the injection apparatus in accordance with an embodiment the invention, the position of a setting element such as a rotational position of a dosing unit may be detected without energy, such as power, in order to ascertain whether a setting element has been altered or not. The passive, non-contact sensor, in accordance with embodiments of the invention, enables a signal to be generated, and a circuit to be activated when the position of a setting element has been altered. Accordingly, the non-contact sensor may detect the change, while no power is consumed if a setting element is not activated or its position is not altered. It is thus unnecessary to generate a signal which has to be processed by a specific evaluation circuit such as an operation amplifier for ascertaining phase angles, which saves space in the apparatus and may reduce the costs and power requirement.

Generating digital signals using the passive, non-contact sensor, such as a magnetic switch or Reed contact, according to some embodiments, may be useful when dosing according to predetermined whole units and not between these predetermined whole units.

Although the invention is described on the basis of an exemplary injection apparatus, the invention is also intended to relate to its use for detecting the position of a setting element in any medical apparatus for dispensing a substance in doses.

According to some embodiments of the invention, two or more passive, non-contact sensors may be arranged on or in an injection apparatus and may be positioned such that two sensors lie on a circle around the rotational axis of a dosing unit. According to this example, the individual passive, non-contact sensors may be arranged such that they are positioned uniformly on the circular line, i.e. the angular distance between any two adjacent sensors is about equal. Alternatively, it may also be possible to arrange the passive, non-contact sensors so that they are distributed non-uniformly, i.e. where two sensors are arranged around the rotational axis of a rotor used for setting, and where the sensors form an angle of about 90° with respect to the rotational axis.

The passive, non-contact sensors, according to one embodiment, may be arranged both lying in a plane and around the central axis of a setting element. Alternatively or in addition, at least one passive, non-contact sensor may be axially offset, i.e. arranged shifted parallel to the rotational axis of the setting element with respect to another passive, non-contact sensor, in order to lie outside a shielding, as described below. At least one passive, non-contact sensor may also be arranged such that a reset signal may be generated once the set dosage has been dispensed.

The passive, non-contact sensors, such as magnetic switches or Reed contacts, according to a further embodiment, may be configured as SMDs (Surface Mounted Devices), such that the sensors may be moulded into a circuit, which may reduce the height of the circuit. This, for example, enables an injection apparatus or pen to be formed with a relatively uniform thickness in the axial direction, as a “camel's hump” in the area of the sensors for ascertaining the rotational position is no longer necessary. Furthermore, by using SMD technology, the passive, non-contact sensors may be moulded with the circuit connected to them, which may create a more robust arrangement and may eliminate corrosion problems. As a result, the sensors may be attached directly on a printed circuit (printed board assembly or folded printed board assembly) and are not required to be assembled separately, which may reduce the manufacturing and assembly costs of an injection or infusion apparatus.

According to certain embodiments, at least one shield may be provided for shielding against interfering magnetic fields. The shield may be arranged around at least one passive, non-contact sensor, in order to shield against external inference fields and to avoid incorrect signals, for example.

The shielding against interfering magnetic fields may, according to some embodiments of the present invention, be provided so that when a number of sensors are used, at least one sensor is still arranged outside the shielding,. The unshielded sensor may be used for identifying errors, because the unshielded sensor responds to inference fields more quickly than the shielded sensors. As a result, an evaluation circuit may identify from a signal of the unshielded sensor that signals outputted by the passive, non-contact sensors, such as a magnetic switch establishing a contact, have been caused by inference fields and are not the result of a setting element being activated.

In accordance with another embodiment, at least one magnetic ring may be connected to the setting element. A magnetic ring, for example, may exhibit an alternating magnetic alignment along its circumference, such that a magnetic north pole and a magnetic south pole are alternately arranged along the circumference of the magnetic ring. The magnetic ring may, for example, be a magnetized plastic ring or a plastic-bonded multi-polar injected ring and may be continuously formed from a material without interruptions or may be formed by individual segments which are attached to each other to form the magnetic ring. An exemplary magnetic ring is shown and described in WO 02/064196 A1, the teaching of which is incorporated herein by reference. One, two, three or more north poles, and an equal number of south poles, for example, may be arranged around the complete circumference of a magnetic ring, wherein the polarities change uniformly in the circumferential direction of the magnetic ring.

The magnetic ring, according to one embodiment, may also be formed such that an alternating polarity, i.e. at least one change between a magnetic north pole and a magnetic south pole, is also provided in the axial direction of the magnetic ring. This may enable an axial shift of the magnetic ring to also be detected using a passive, non-contact sensor, in order to identify whether or not a substance has been completely delivered or dispensed. In addition or alternatively, another magnetic ring may also be arranged on the setting element, axially offset, in order to generate a reset signal once the set dosage has been dispensed.

An additional magnetic ring, according to a further embodiment, may be provided in or on an injection device in accordance with the invention. The ring may be axially offset with respect to the magnetic ring connected to the setting element, and the polarity distribution or the number and distance of the pole changes of the first magnetic ring may correspond to the number and distance of the pole changes of the second magnetic ring. This may, for example, cause a rotatable setting element connected to a magnetic ring to only be stable in particular rotational positions determined by its co-operation with a magnetic ring connected to the injection apparatus. This may serve as a latching function using two magnetic rings lying axially offset with respect to each other, wherein a rotatable setting element may “latch” in those positions in which the magnetic poles of the first magnetic ring oppose the corresponding counter poles of the second magnetic ring and wherein the position of the setting element is unstable between these positions. Alternatively, instead of a magnetic ring, a shaped piece of punched iron may also be used, for example an iron sheet made of a material such as those used for metal stator sheets in the field of motors. This may realize a magnetic latching, in particular given a suitable polarity of the magnetization used.

In a further embodiment, at least one passive, non-contact sensor for generating signals for detecting the position of a setting element and a circuit having an open state and closed state is provided. The circuit, when in an open state is considered to be in an initial position, and in a closed state is considered to be in a second position. The circuit changes to a second position upon a change in the at least one passive, non-contact sensor. The circuit may be off when in an open state, and off on when in a closed state, and may move between ON and OFF via a switch.

Two or more passive, non-contact sensors may be provided with an injection apparatus in accordance with the present invention, which may be arranged on or in the injection apparatus on a circle around a rotational axis of the setting element. The sensors may be arranged uniformly on a circular line around the rotational axis of the setting element, or may be arranged non-uniformly around the rotational axis. In another example, the sensors may be arranged axially offset around the rotational axis of the setting element.

According to another embodiment of the present invention, the at least one passive, non-contact sensor of the injection apparatus may be produced using SMD technology.

In another embodiment, the injection apparatus includes a magnetic ring coupled to the setting element. The magnetic ring may have a change in magnetization in one or more instances in the circumferential direction of the rotational axis of the setting element. Rotating the setting element results in a change in the circuit due to the changing magnetization. In addition, the magnetization of the magnetic ring changes at least once in the axial direction of the magnetic ring. In a further embodiment, a second magnetic ring may be provided and may be disposed on the injection apparatus. The first and second rings may be axially offset. In addition, the number and a distance of pole changes of the first magnetic ring may correspond to a number and a distance of pole changes of said second magnetic ring. In some embodiments, the first and second magnetic ring may act together as a latch, so that when the rings are in an opposing position, i.e. opposing magnetization or polarity, the rings latch. When the poles of the rings are between an opposing position, they are unstable. The magnetic rings may be formed of any suitable magnetic or magnetized material, which may include one or more punched iron pieces.

In yet another embodiment of the present invention, an injection apparatus is provided with a plurality of passive, non-contact magnetic sensors having an open and closed state, and a magnetic ring coupled to a setting element of the injection apparatus. The magnetic field of the magnetic ring has a first strength at one or more north poles and south poles and a second strength between the poles. The passive, non-contact sensors may be responsive to the magnetic field of the magnetic ring in areas having the first strength. In a further example, a second magnetic ring is provided that has a magnetic field offset from said first magnetic ring. In response to a delivery motion of the injection apparatus, the second ring opposes the sensors and causes the sensors to generate a reset signal which may cause an LCD display to reset a dosage reading, for example.

In another embodiment, an injection apparatus includes a plurality of passive, non-contact magnetic sensors having an open and closed state, a shielding coupled to at least one of the plurality of magnetic switches, where at least another one of the plurality of magnetic switches is not coupled to said shielding and is unshielded. A magnetic ring is also provided and is coupled to a setting element of said injection apparatus having a magnetic field of a first strength at one or more north poles and south poles and a second strength between the poles. The passive, non-contact magnetic are responsive to the magnetic field of the first magnetic ring having the first strength. In addition, an evaluation unit is provided for sensing when said plurality of passive, non-contact magnetic sensors are responsive to the magnetic ring. The evaluation unit also receives a signal from one or more shielded sensors and from the unshielded sensor in order to identify sensor error. In addition the evaluation unit determines angular position of the magnetic ring using sensor signals.

### BRIEF DESCRIPTION OF THE DRAWINGS

FIG. 1 is a schematic diagram of a magnetic ring which can be attached to a setting element, comprising passive, non-contact sensors in accordance with the invention, in accordance with one embodiment;

FIG. 2 is a schematic diagram of a segment piece of a magnetic ring;

FIG. 3 is a diagram of an injection apparatus in accordance with the invention, with an ampoule placed on it, before a substance is dispensed;

FIG. 4 is a diagram of an injection apparatus in accordance with the invention, without an ampoule, after a substance has been dispensed;

FIG. 5 is a block circuit diagram to illustrate the functionality of the injection apparatus in accordance with the invention;

FIGS. 6a and 6b is another embodiment of the arrangement of magnets and passive, non-contact sensors in an injection apparatus; and

FIG. 7 depicts sensor signals generated by the arrangement shown in FIGS. 6a and 6b.

### DETAILED DESCRIPTION OF THE DRAWINGS

With regard to fastening, mounting, attaching or connecting the components of devices of the present invention, unless specifically described as otherwise, conventional fasteners such as screws, rivets, toggles, pins and the like may be used. Other fastening or attachment means appropriate for connecting components include friction fitting, adhesives, welding and soldering, the latter particularly with regard to electrical or processing components or systems of the devices. Any suitable electronic, electrical, communication, computer or processing components may be used, including any suitable electrical components and circuitry, wires, wireless components, sensors, chips, boards, micro-processing or control system components, software, firmware, hardware, etc.

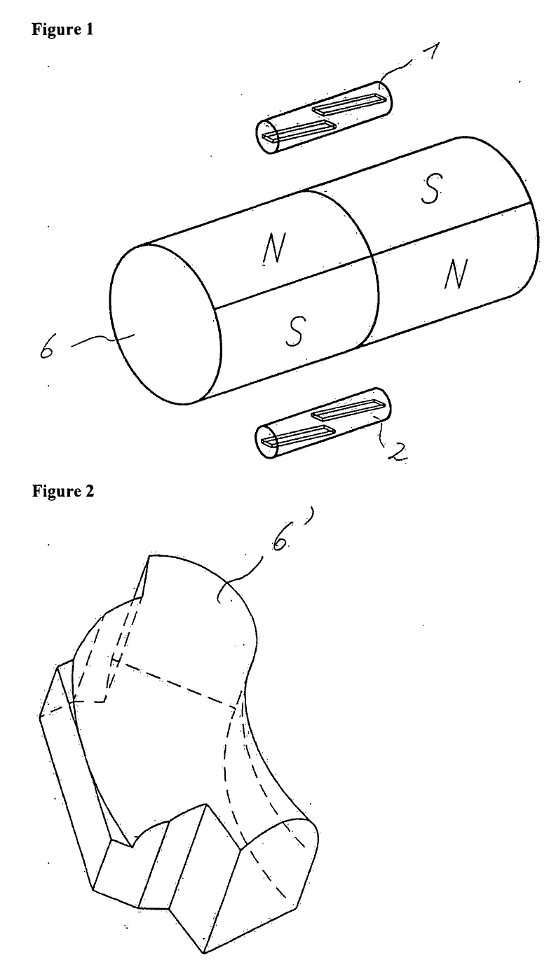

FIG. 1 schematically shows a magnetic ring 6 connected to a rotatable setting element (not shown), in which the magnetic polarity changes both from N. to S. and vice versa in the circumferential direction of the magnetic ring 6 and wherein such a change in polarity is also provided in the axial direction of the magnetic ring 6. In the exemplary embodiment, two magnetic switches 1 and 2 serving as passive, non-contact sensors are arranged around the magnetic ring 6 and are connected to the injection apparatus (not shown). If the magnetic ring 6 connected to the setting element is then rotated or shifted in the axial direction, then the magnetic switches 1, 2 always close when a particular strength of the magnetic field is exceeded, i.e. when for example a magnetic north pole or a magnetic south pole enters the immediate vicinity of one of the magnetic switches. In the area of the change in polarization between a magnetic north pole and a magnetic south pole, the magnetic field strength drops, such that a magnetic switch opens. If the magnetic switches 1, 2 are suitably arranged, then rotating the magnetic ring 6 connected to a setting element generates a rectangular signal from each magnetic switch 1, 2, wherein the angular position of the magnetic ring 6 and therefore of the setting element in the injection apparatus may be ascertained from the combination of such rectangular signals from two or more magnetic switches. An axial shift of the magnetic ring 6 may also be detected analogously.

FIG. 2 shows a segment piece 6′ of a magnetic ring that may be used in accordance with the invention, where a magnetic ring having a polarity which changes in the circumferential direction can be manufactured from a number of segment pieces which engage with each other. In additional embodiments, other types of segment shapes may be used to manufacture a magnetic ring. Each piece or segment may be composed of any suitable material, for example, any suitable magnetic, electromagnetic or magnetic field producing material, magnetized plastic, multi-polar injected materials, or may be a punched iron piece.

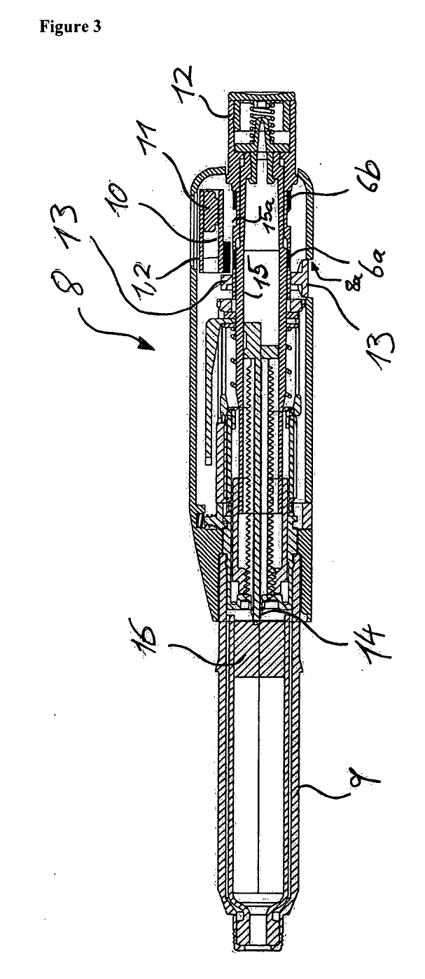

FIG. 3 shows an injection apparatus or pen 8 with an ampoule 9 and dosing button 12 which is rotatably mounted in the injection apparatus 8 and connected to a drive member 15. A first magnetic ring 6a for detecting a dosage set and a second magnetic ring 6b for generating a reset signal are arranged, axially offset with respect to each other, around the drive member 15. In the initial position shown in FIG. 3, the magnetic ring 6a opposes the Reed contacts 1, 2 attached to the injection apparatus 8, such that a rotation at the dosing button 12 results in a rotation of the magnetic ring 6a which is connected to the dosing button 12 via the drive member 15, which may be detected using the signals generated by the Reed contacts 1, 2. The signals generated by the Reed contacts 1, 2 are processed by a printed circuit 10 and converted into signals for the LCD display 11, such that a dosing set at the dosing button 12 may be read on the LCD display 11.

The rotation of the dosing button 12 results, for example via threaded engagement, in an axial shift of the threaded rod 14 which is mounted such that it cannot be rotated, which enables the size or length of the shift of the stopper 16 in the ampoule 9 to be set in a known way.

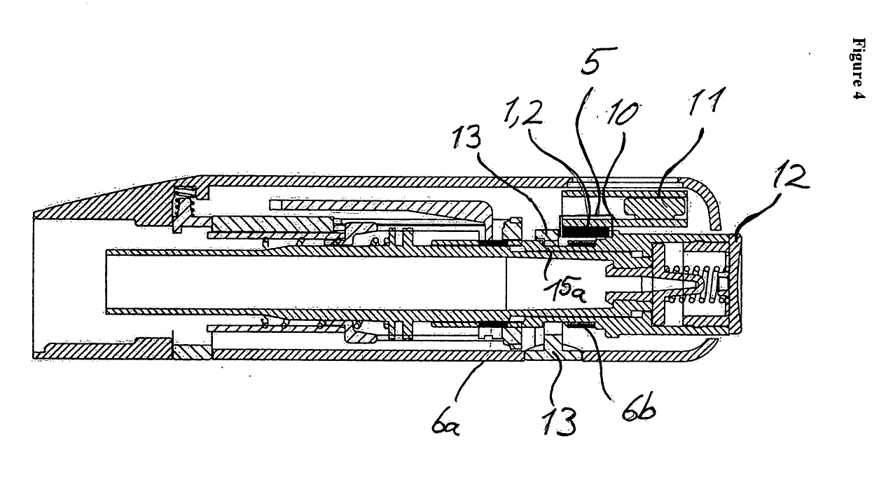

If the dosing has been set as desired by the dosing button 12, then the dosing button 12 is pressed into the injection apparatus 8, dispensing the desired dosage of a substance contained in the ampoule 9 in a known way. As shown in FIG. 4, this advances the magnetic ring 6b, which is offset backwards with respect to the magnetic ring 6a in the axial direction from the dispensing end, towards the dispensing side of the injection apparatus 8, such that the magnetic ring 6b opposes the Reed contacts 1, 2, which enables a reset signal to be generated, to reset the dosage set on the LCD display 11.

FIG. 4 shows an embodiment comprising a shielding 5 of the Reed contacts 1, 2 which may optionally be provided.

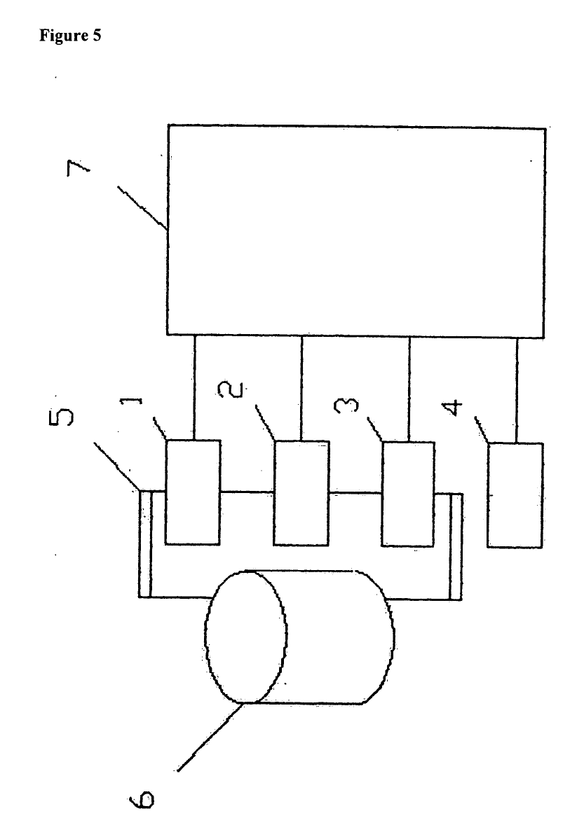

The reset switch ring 13 shown in an unlocked position in FIG. 3 abuts the drive member 15 in the position shown in FIG. 3 and is biased such that when the drive member 15 is shifted towards the dispensing opening by activating the dosing button 12, the reset switch ring 13 latches into the groove 15a of the drive member 15, such that the part of the reset switch ring 13 which may be moved through a cavity or opening 8a of the injection apparatus 8 is moved radially outwards, as shown in FIG. 4. Pressing the reset switch ring 13 releases the lock between the drive piece 15 and the reset switch ring 13, such that the drive piece 15 may be slid back to the initial position shown in FIG. 3, for example by a spring force. Although the magnetic rings 6a and 6b, shielding 5, and Reed contacts 1 and 2, are located distally with respect to the ampoule-end of the injection apparatus 8 in FIGS. 4 and 5, the magnetic rings, shielding, and Reed contacts may be positioned proximal to the injection apparatus ampoule-end or in any suitable location in or on the injection apparatus 8.

FIG. 5 shows a block circuit diagram of the parts of an injection apparatus relevant for the invention. A magnetic ring 6 which is polarized as described above is connected to a setting element, wherein the rotational position of the magnetic ring 6 is detected by the Reed contacts 1 and 2. Another Reed contact 3 is provided for a reset function, to detect the position of the magnetic ring 6b shown in FIGS. 3 and 4, such that an active reset signal is generated when a dosing button has been completely pressed. The Reed contacts 1, 2 and 3 are situated within a shielding 5 and are thus protected from interfering magnetic fields. Another Reed contact 4 is arranged outside the shielding 5, such that it responds to interfering magnetic fields more easily than the Reed contacts 1, 2 and 3, in order to then generate an error signal and to avoid malfunctions due to misinterpreting the signals outputted by the Reed contacts 1 to 3. An evaluation unit or “E module” 7 is connected to the Reed contacts 1 to 4 and receives and processes and/or evaluates the signals generated by the Reed contacts 1 to 4 and outputs them to a display device. The signals generated by the Reed contacts 1 to 4 are digital signals, i.e. the Reed contacts 1 to 4 are only closed when magnetic fields which are above a magnetic field strength which may be predetermined are applied to the respective Reed contacts, wherein the magnetic ring 6 and the Reed contacts 1 to 4 may be arranged such that in the resting state, in which a setting process is not performed, all the Reed contacts 1 to 4 are open and thus no power is consumed. By closing at least one of the Reed contacts 1 to 4, the evaluation unit 7 activated, enabling the power consumption of the apparatus as a whole to be reduced.

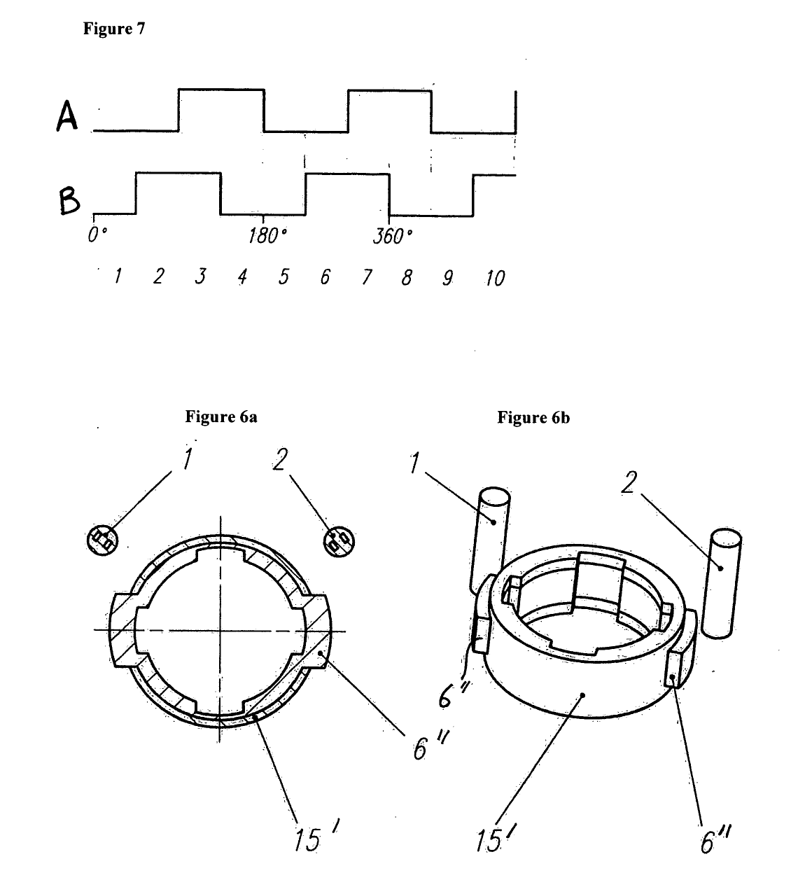

FIGS. 6a and 6b show another exemplary embodiment of a device for identifying a rotational position of a setting element in an injection apparatus. Magnets 6” are provided on each of opposing sides of the rotatably mounted setting element 15′. Two magnetic switches 1 and 2, used as passive, non-contact sensors, are arranged at a predetermined distance away from the setting element 15′, such that when the setting element 15′ is rotated, the magnets 6″ are guided past the magnetic switches 1, 2.

FIG. 7 shows the digital signals A and B outputted by the magnetic switches 1, 2, wherein from the sequence of these signals, it is possible to identify how the setting element 15′ has been rotated relative to the magnetic switches 1, 2 and therefore relative to the injection apparatus. In the exemplary embodiment shown, eight different states may be distinguished within one complete revolution of 360°, i.e. a rotation of 45° may be detected.

Embodiments of the present invention, including preferred embodiments, have been presented for the purpose of illustration and description. They are not intended to be exhaustive or to limit the invention to the precise forms or steps disclosed. The embodiments were chosen and described to provide the best illustration of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. An injection apparatus comprising at least one passive, non-contact sensor which can generate signals for detecting the position of a setting element.

2. The injection apparatus as set forth in claim 1, wherein the at least one passive, non-contact sensor comprises a magnetic switch or Reed contact.

3. The injection apparatus as set forth in claim 1, wherein said at least one passive, non-contact sensor comprises two or more passive, non-contact sensors.

4. The injection apparatus as set forth in claim 1, wherein the at least one passive, non-contact sensor is an SMD sensor.

5. The injection apparatus as set forth in claim 1, further comprising a shielding for the at least one passive, non-contact sensor.

6. The injection apparatus as set forth in claim 5, wherein the at least one sensor lies outside the shielding.

7. The injection apparatus as set forth in claim 1, further comprising at least a first magnetic ring which is coupled to the setting element, wherein the magnetization of the first magnetic ring changes at least once in the circumferential direction.

8. The injection apparatus as set forth in claim 7, further comprising one of a second magnetic ring or an FE punched bent part coupled to the injection apparatus.

9. The injection apparatus as set forth in claim 1, further comprising a magnetic ring, wherein the magnetization of the magnetic ring changes at least once in the axial direction of the magnetic ring.

10. The injection apparatus as set forth in claim 9, further comprising one of a second magnetic ring or an FE punched bent part coupled to the injection apparatus.

11. An injection apparatus comprising:
at least one passive, non-contact sensor for generating signals for detecting the position of a setting element;
a circuit having an open state and a closed state, wherein said circuit is in an open state when said circuit is in an initial position, said circuit in a closed state upon a change from said initial position of said at least one passive, non-contact sensor to a second position.

12. The injection apparatus as set forth in claim 11, wherein said circuit comprises a measuring circuit, said measuring circuit switching on when in a closed state and switching off when in an open state.

13. The injection apparatus as set forth in claim 11, wherein said at least one passive, non-contact sensor comprises two or more passive, non-contact sensors.

14. The injection apparatus as set forth in claim 13, wherein said sensors are arranged on or in the injection apparatus on a circle around a rotational axis of the setting element.

15. The injection apparatus as set forth in claim 14, wherein said sensors are arranged uniformly on a circular line around the rotational axis of the setting element.

16. The injection apparatus as set forth in claim 14, wherein said sensors are arranged non-uniformly on a circular line around the rotational axis of the setting element.

17. The injection apparatus as set forth in claim 14, wherein said sensors are arranged axially offset around the rotational axis of the setting element.

18. The injection apparatus as set forth in claim 11, wherein the at least one passive, non-contact sensor is produced using SMD technology such that one or more of the at least one passive, non-contact sensor is mounted with said circuit.

19. The injection apparatus as set forth in claim 11, further comprising at least a first magnetic ring which is coupled to the setting element, wherein the magnetization of the first magnetic ring changes at least once in the circumferential direction, and wherein upon adjusting said setting element in the circumferential direction, said circuit changes state.

20. The injection apparatus as set forth in claim 19, further comprising a second magnetic ring which is coupled to the injection apparatus.

21. The injection apparatus as set forth in claim 20, wherein said first and second magnetic ring are axially offset.

22. The injection apparatus as set forth in claim 20, wherein a number and a distance of pole changes of the first magnetic ring correspond to a number and a distance of pole changes of said second magnetic ring.

23. The injection apparatus as set forth in claim 20, wherein said first and second magnetic rings can latch when poles of each of said first and second magnetic ring are in an opposing position, and wherein said rings are unstable when the poles of each of said first and second magnetic ring are between the opposing position.

24. The injection apparatus as set forth in claim 20, wherein said second magnetic ring comprises one or more punched iron pieces.

25. The injection apparatus as set forth in claim 11, further comprising a magnetic ring, wherein the magnetization of the magnetic ring changes at least once in the axial direction of the magnetic ring.

26. The injection apparatus as set forth in claim 25, further comprising one of a second magnetic ring or one or more shaped iron pieces forming a ring connected to the injection apparatus.

27. An injection apparatus comprising:
a plurality of passive, non-contact magnetic sensors having an open and closed state; and
a first magnetic ring coupled to a setting element of said injection apparatus, and having a magnetic field, wherein said magnetic field has a first strength at one or more north poles and south poles and a second strength between said poles, said plurality of passive, non-contact sensors being responsive to the magnetic field of said first magnetic ring in one or more areas having the first strength.

28. The injection apparatus of claim 27, further comprising a second magnetic ring having a magnetic field offset from said first magnetic ring, wherein upon a delivery motion of said injection apparatus, said second ring opposes said sensors and said sensors generate a reset signal.

29. The injection apparatus of claim 28, wherein said reset signal causes an LCD display to reset a dosage reading.

30. A delivery device for administering a substance, the delivery device comprising an operational mechanism and at least one passive, non-contact sensor for sensing a position associated with the operational mechanism.

31. The delivery device according to claim 30, wherein the at least one sensor comprises at least two passive, non-contact magnetic sensors.

32. The delivery device according to claim 31, further comprising at least a first magnetic body coupled to the operational mechanism, wherein the magnetization of the first magnetic body changes at least once in a selected direction relative to the magnetic body.

33. The delivery device according to claim 32, further comprising one of a second magnetic body or an FE punched bent part coupled to the delivery device.

34. An injection apparatus comprising:
a plurality of passive, non-contact magnetic sensors having an open and closed state;
a shielding coupled to at least one of the plurality of magnetic sensors, wherein at least another one of the plurality of magnetic sensors is not coupled to said shielding and is unshielded;
a first magnetic ring coupled to a setting element of said injection apparatus, and having a magnetic field, wherein said magnetic field has a first strength at one or more north poles and south poles and a second strength between said poles, said plurality of magnetic sensors being responsive to the magnetic field of said first magnetic ring having the first strength; and
an evaluation unit for sensing that said plurality of magnetic sensors are responsive to said first magnetic ring, said evaluation unit comparing a response signal from one or more shielded sensors with a response signal from the unshielded sensor in order to identify sensor error.

35. The injection apparatus of claim 34, wherein said evaluation unit determines angular position of said magnetic ring using sensor response signals.
